(19) United States
(12) Patent Application Publication
(10) Pub. No.: US 2023/0242996 A1
(43) Pub. Date: Aug. 3, 2023

(54) NOVEL TUMOR MARKER FOR HEPATOCELLULAR CARCINOMA AND CLINICAL APPLICATION THEREOF

(71) Applicant: The First Affiliated Hospital of Sun Yat-Sen University, Guangzhou (CN)

(72) Inventors: Jiaping Li, Guangzhou (CN); Jie Wen, Guangzhou (CN); Xiaokai Zhang, Guangzhou (GD)

(73) Assignee: The First Affiliated Hospital of Sun Yat-Sen University, Guangzhou (CN)

(21) Appl. No.: 18/075,464

(22) Filed: Dec. 6, 2022

(30) Foreign Application Priority Data
Apr. 21, 2022 (CN) 202210422869.X

(51) Int. Cl. C12Q 1/6886; C12Q 1/686; C12Q 1/6806

(52) U.S. Cl. CPC C12Q 1/6886; C12Q 1/686; C12Q 1/6806; C12Q 2600/158; C12Q 2600/118

(57) ABSTRACT

The present disclosure relates to a newly discovered tumor marker for hepatocellular carcinoma (HCC) and related applications thereof in clinical detection. The tumor marker for HCC in the present disclosure is OIT3, which is abnormally expressed at low levels in HCC tissues and can be used in the diagnosis and treatment of HCC. The present application further relates to primer sequences of OIT3 as an HCC marker, which can be used as a reagent for the detection and diagnosis of HCC. The present disclosure can improve the accuracy, efficiency, and diagnosis of early HCC and is suitable for popularization in the clinical diagnosis of HCC.

### REFERENCE TO SEQUENCE LISTING

The sequence listing is submitted as a XML file filed via EFS-Web, with a file name of “Sequence_Listing_GZYR-U.S. Pat. No. 1,223,848-97-1796.XML”, a creation date of Apr. 22, 2023, and a size of 3467 bytes. The sequence Listing filed via EFS-Web is a part of the specification and is incorporated in its entirety by reference herein.

### TECHNICAL FIELD

The present disclosure relates to the tumor technical field of hepatocellular carcinoma (HCC), in particular to a novel tumor marker for HCC and clinical application thereof.

### BACKGROUND

Liver cancer is the fifth most common cancer worldwide, and its mortality rate is the second highest cancer-related mortality rate worldwide. Approximately 854,000 new cases of liver cancer are diagnosed annually, and approximately 810,000 deaths due to liver cancer are also recorded yearly. HCC accounts for over 90% of newly diagnosed liver cancer cases worldwide. The proportion of newly diagnosed cases in Asia is as high as 75-80%, especially in China, where the number of newly diagnosed cases of HCC accounts for more than 50% of the total cases in Asia. Early detection and treatment are key for the prevention and treatment of HCC. Because of the lack of obvious discomfort symptoms in the early stages, most patients are not diagnosed with early-stage lesions. Patients are often diagnosed at advanced stages when there are obvious clinical symptoms, poor liver function, and loss of surgical opportunity. Currently, the diagnosis of HCC mainly depends on imaging and the detection of elevated serum alpha-fetoprotein (AFP) levels.

However, the sensitivity of imaging modalities for HCC lesions smaller than 2 cm is poor, which is related to incomplete tumor vascular structure. Since the 1970s, serum AFP detection has been used for the clinical diagnosis of HCC, improving the accuracy of diagnosis, especially in patients with advanced HCC who have clinical symptoms. The use of serum AFP detection has a sensitivity of 39-64% and a specificity of 76-91%. Even in cases of early HCC, AFP detection is still beneficial. Currently, AFP detection is widely used for the clinical diagnosis of HCC. However, 80% of patients with HCC still have no significant change in peripheral blood AFP. For the diagnosis of HCC lesions >3 cm, the sensitivity of AFP detection can be up to 52%, whereas for the diagnosis of HCC lesions <3 cm, the sensitivity of AFP detection is only 25%. Additionally, some patients with HCC are AFP-negative (AFP<25 ng/mL). Furthermore, a significant increase in AFP levels can also appear in patients without HCC, such as those with acute hepatitis. In summary, more accurate, sensitive, reliable, and widely applicable diagnostic indicators are needed to diagnose HCC.

Surgery is a major treatment option for patients with HCC. Patients with early HCC can be treated surgically, with a 5-year survival rate of 70%, while patients with advanced HCC can only be treated conservatively, as they have a poor clinical prognosis with a median survival time of less than 1 year. For patients with moderate or advanced HCC, multidisciplinary comprehensive treatments, such as interventional embolization, local ablation, and local radiotherapy, are also required. Even after multidisciplinary treatment, the long-term prognosis of patients with HCC remains very poor. Furthermore, the postoperative recurrence rate of HCC is high, recurrence often occurs early, and there is no specific treatment. Targeted therapy and immunotherapy can have some clinical benefits but still cannot achieve higher treatment efficiency. Due to the above-mentioned facts, we were compelled to examine the molecular biological basis of HCC, such as signaling regulation pathways involved in the occurrence and development, invasion and metastasis, resistance to radiotherapy and chemotherapy, and recurrence of HCC, to discover novel tumor markers for HCC, provide new ideas for early diagnosis and prognostic assessment of HCC, and improve the level of diagnosis and treatment of HCC in China.

Based on this, the present disclosure provides a novel tumor marker for HCC and clinical application thereof.

### SUMMARY

In view of the defects of the prior art, an object of the present disclosure is to provide a novel tumor marker for HCC and application thereof, so as to solve the problems raised in the above-mentioned section of Background Art.

In order to solve the technical problems, the present disclosure provides the following technical solutions:

The present disclosure provides a novel tumor marker for HCC, wherein the tumor marker for HCC is OIT3, and a level of expression of the OIT3 in peripheral blood and HCC tissues of patients with HCC is detected using qRT-PCR;

wherein the OIT3 is expressed at significantly low levels in HCC tissues and is closely related to tumor biological behaviors of hepatoma cells.

Preferably, the tumor biological behaviors are proliferation, migration, and invasion.

Preferably, the tumor marker for HCC, OIT3, is expressed at low levels in HCC tissues, and the expression level thereof is significantly related to the clinical prognosis of patients with HCC.

Preferably, the tumor marker may be used as a key component of an HCC detection kit by designing primer sequences, and clinical samples of patients with HCC can be detected as tumor markers.

Preferably, the clinical samples comprise specimens of peripheral blood and HCC tissues, and relevant samples should be collected clinically. The collection procedures of peripheral blood samples are as follows:

Collecting 2.5-3.5 mL of fasting venous blood from subjects into a pro-coagulation tube, and keeping the same vertically at 25° C.±5° C. for 25-35 min, followed by centrifugation for 8-12 min at 1450-1550 r/min at normal temperature, then transferring the supernatant into a marked eppendorf (EP) tube and saving same at −80° C. for future use.

Preferably, the collection procedures for the HCC specimens are as follows: fresh HCC tissue samples were obtained and stored at −80° C. for subsequent extraction of total RNA from serum samples and HCC tissue samples with TRIzol reagent.

Preferably, primer sequences of the OIT3 are as follows:

Forward Primer:
(SEQUENCE ID NO: 1)
TGTTCTGCTTACATCAGCCTG;
Reverse Primer:
(SEQUENCE ID NO: 2)
GTTGTCACATAGAGGAGGACCT

The levels of expression of the OIT3 in HCC tissue samples and peripheral blood of patients are measured with fluorescence quantitative PCR (qRT-PCR) using specific primers.

Preferably, provided is the application of the tumor marker for HCC as an HCC detection and diagnosis reagent and the value in a prediction model for clinical prognosis.

The present disclosure further provides an application of the detection of the HCC tumor marker in the diagnosis of HCC.

Preferably, the application may include an early screening of patients with HCC and prediction of clinical prognosis based on the levels of expression of the OIT3, which has clinical transformation value.

Compared with the prior art, the present disclosure has the following beneficial effects:

1. The present disclosure provides a tumor marker for HCC that can be used as a diagnostic reagent for HCC and applied in the early diagnosis of patients with HCC, making the diagnosis of patients with HCC simpler, accurate, and rapid while improving the diagnosis and treatment of HCC in China, is suitable for preliminary screening of clinical patients, and can be popularized and applied.

2. The present disclosure further provides the primer sequences of the above-mentioned marker for HCC, which can be used as an important component of the OIT3 detection kit, thus making the detection of the tumor marker for HCC simpler and rapid, practical, and convenient for wider application.

3. The present disclosure further provides a prediction model for the detection level of the tumor marker and the clinical prognosis of patients to provide some ideas for the judgment of clinical outcomes of patients during the clinical diagnosis and treatment of HCC, which is thus useful for the diagnosis and treatment of HCC.

### BRIEF DESCRIPTION OF THE DRAWINGS

FIG. 1 shows a schematic diagram for verifying the difference in OIT3 expression between HCC tissues and normal paracancerous tissues in different tumor databases.

FIG. 2 shows a schematic diagram for verifying the difference in OIT3 expression in HCC and paracancerous tissues using immunohistochemistry in clinical samples (tissue microarray) of HCC.

FIG. 3 shows a schematic diagram for detecting the mRNA expression level of OIT3 in hepatoma cells and hepatocytes.

FIG. 4 shows a schematic diagram for verifying the relationship between the expression level of OIT3 and the clinical prognosis of patients with HCC in the database and tissue microarray.

FIG. 5 shows a schematic diagram for verifying the ability of OIT3 to inhibit the proliferation, invasion, and metastasis of hepatoma cells.

FIG. 6 shows a schematic diagram for exploring the regulatory mechanism of OIT3 in HCC and the changes in arachidonic acid.

### DETAILED DESCRIPTION OF THE EMBODIMENTS

Referring to the figures for Examples of the present disclosure, the technical solutions described in the examples of the present disclosure are clearly and completely described below. Obviously, the examples described are only a part of the examples of the present disclosure and are not all the examples thereof. All other examples that persons of ordinary skill in the art obtained without creative efforts based on the examples of the present disclosure also fall within the scope of the present disclosure.

In order to make the diagnosis of patients with HCC simpler, convenient, rapid, and accurate, the present disclosure provides a novel tumor marker for HCC, that is, OIT3. Furthermore, as a tumor marker, we have designed primers for fluorescence quantitative PCR to detect the expression level of OIT3 in hepatoma cells. In addition, the present disclosure verifies the correlation between the OIT3 expression level and the clinical prognosis of patients with HCC. The present disclosure further verifies that OIT3 may have inhibitory activity against cancer in HCC tissues in terms of tumor cell function. The present disclosure provides the application for the detection of OIT3 as a tumor marker for HCC and the application of real-time quantitative PCR primers for OIT3 in diagnostic reagents for HCC.

According to an example of the present disclosure, provided is a novel tumor marker for HCC, wherein the tumor marker for HCC is OIT3, and the expression level of the OIT3 in peripheral blood and HCC tissues of patients with HCC is detected using qRT-PCR;

wherein the OIT3 is expressed at a significantly low level in HCC tissues and is closely related to tumor biological behaviors of hepatoma cells.

According to an example of the present disclosure, the tumor biological behaviors are proliferation, migration, and invasion.

According to an example of the present disclosure, the tumor marker for HCC, OIT3, is expressed at low levels in HCC tissues, and its level of expression is significantly related to the clinical prognosis of patients with HCC.

According to an example of the present disclosure, the tumor marker may be used as a key component of an HCC detection kit by designing primer sequences, and clinical samples of patients with HCC can be tested for the tumor marker.

According to an example of the present disclosure, clinical samples comprise specimens of peripheral blood and HCC tissues, and relevant samples should be collected clinically, wherein the collection procedures for peripheral blood samples are as follows:

Collecting 2.5-3.5 mL of fasting venous blood from subjects into a pro-coagulation tube, keeping the same vertically at 25° C.±5° C. for 25-35 min, followed by centrifugation for 8-12 min at 1450-1550 r/min at normal temperature, then the supernatant liquid into a marked EP tube and at −80° C. for future use.

According to an example of the present disclosure, the collection procedures of the HCC specimen are as follows: cutting fresh HCC tissues and storing same at −80° C. for subsequent extraction of total RNA from serum samples and HCC tissue samples using TRIzol reagent.

According to an example of the present disclosure, the primer sequences of OIT3 are as follows:

Forward Primer:
(SEQUENCE ID NO: 1)
TGTTCTGCTTACATCAGCCTG;
Reverse Primer:
(SEQUENCE ID NO: 2)
GTTGTCACATAGAGGAGGACCT

and levels of expression of the OIT3 in HCC tissues and peripheral blood specimens of patients are detected using fluorescence quantitative PCR (qRT-PCR) using specific primers.

According to an example of the present disclosure, provided is the application of the tumor marker for HCC as an HCC detection and diagnosis reagent and its value in a prediction model for clinical prognosis.

According to an example of the present disclosure, provided is an application for the detection of tumor markers for HCC in the diagnosis of HCC.

According to an example of the present disclosure, the application may include early screening of patients with HCC and prediction of clinical prognosis based on the levels of expression of OIT3, which has clinical transformation value.

### Example 1

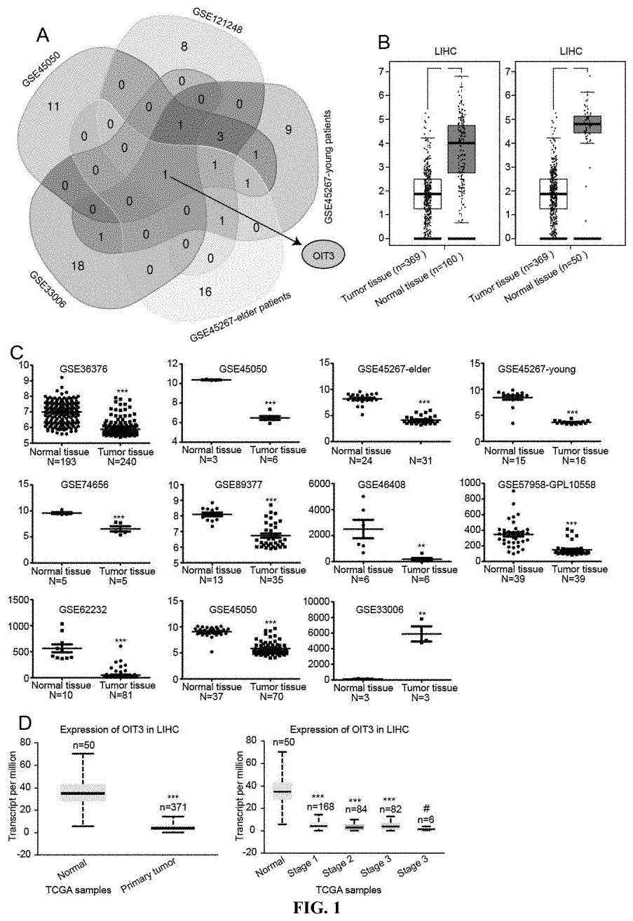

Reference is made to FIGS. 1-6.

FIG. 1 shows a schematic diagram for verifying the difference in OIT3 expression in HCC tissues and normal paracancerous tissues in different tumor databases. It can be seen that OIT3 is generally expressed at low levels in HCC tissues.

FIG. 1: Different databases verify that the level of expression of OIT3 in HCC tissues is significantly lower than that in paracancerous tissues.

A: GSE3306, GSE45050, GSE121248, and GSE45267 in the GEO database (https://www.ncbi.nlm.nih.gov/gds#) are used for intersection analysis.

B: The GEPIA database (http://gepia.cancer-pku.cn/) suggests that the expression of OIT3 in HCC tissues is significantly lower than that in tumor-adjacent tissues.

C: Different GSE datasets suggest that the level of expression of OIT3 in HCC tissues is significantly lower than in paracancerous tissues (except for GSE33006).

D: The UALCAN database (http://ualcan.path.uab.edu/) suggests that the level of expression of OIT3 in HCC tissues is significantly lower than that in paracancerous tissues (*P<0.05; **P<0.01; ***P<0.001).

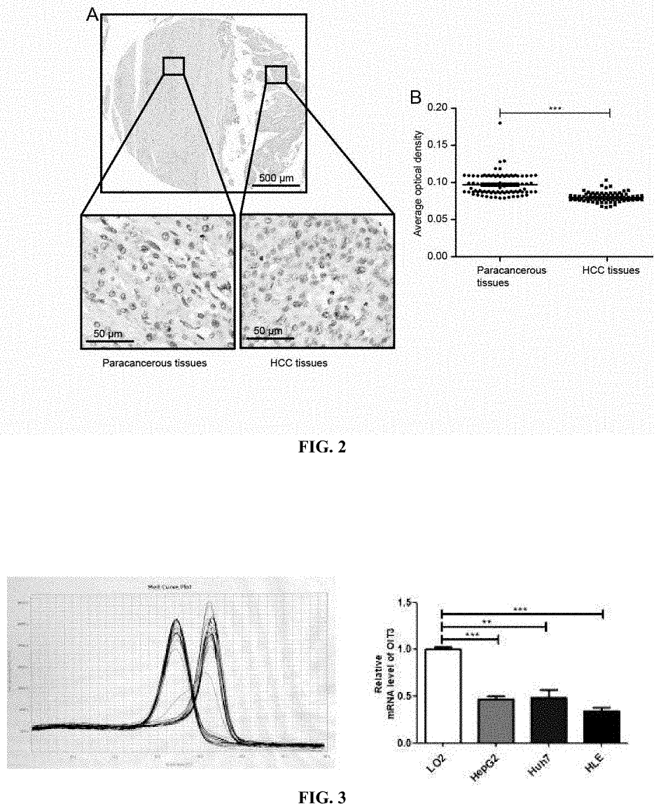

FIG. 2 shows a schematic diagram for verifying the difference in OIT3 expression in HCC tissues and paracancerous tissues using immunohistochemistry in clinical samples (tissue microarray) of HCC. It can be seen that OIT3 is indeed expressed at low levels in HCC.

The results of the HCC tissue microarray confirm that the level of expression of OIT3 in HCC tissues is significantly lower than that in paracancerous tissues; A: representative diagram of immunohistochemistry of OIT3 in HCC tissues and corresponding paracancerous tissues; B: statistical diagram of OIT3 immunohistochemical score in HCC tissues and corresponding paracancerous tissues (***P<0.001).

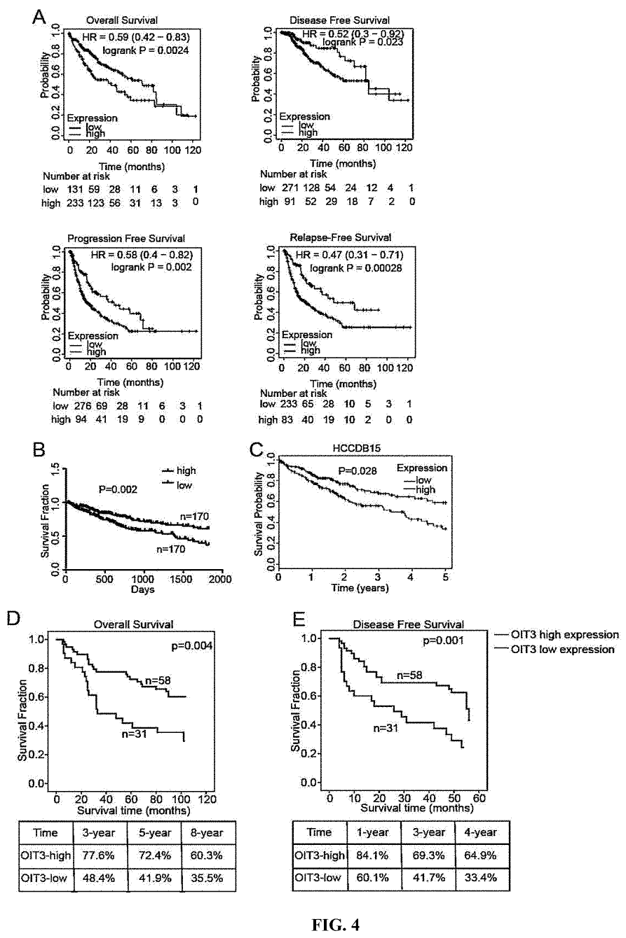

FIG. 3 shows the detection of the mRNA expression level of OIT3 in hepatoma cells and hepatocyte cells, and it can be seen that the level of expression of OIT3 in hepatoma cells is lower than that in hepatocytes, and the level of expression of OIT3 in hepatoma cells is significantly lower than that in normal hepatocytes.

Left panel: Dissolution curves corresponding to OIT3 and GAPDH. It can be seen that both dissolution curves have a single peak, indicating that the primer for OIT3 is correct.

Right panel: Difference in OIT3 mRNA levels in hepatoma cells and hepatocytes (**P<0.01; ***P<0.001).

FIG. 4 shows a schematic diagram for verifying the relationship between the level of expression of OIT3 and the clinical prognosis of patients with HCC in the database and tissue microarray, which reveals a lower level of expression of OIT3 indicates a worse prognosis.

A-C: Different databases suggest that the level of expression of OIT3 is lower and the prognosis of patients is worse (A-B: KM-plotter: http://kmplot.com/analysis/index.php!p=service;

C: HCCDB: lifeome.net/database/hccdb/home.html); D-E: the results of tissue microarray suggest that patients who have relatively lower levels of expression of OIT3 have worse prognoses.

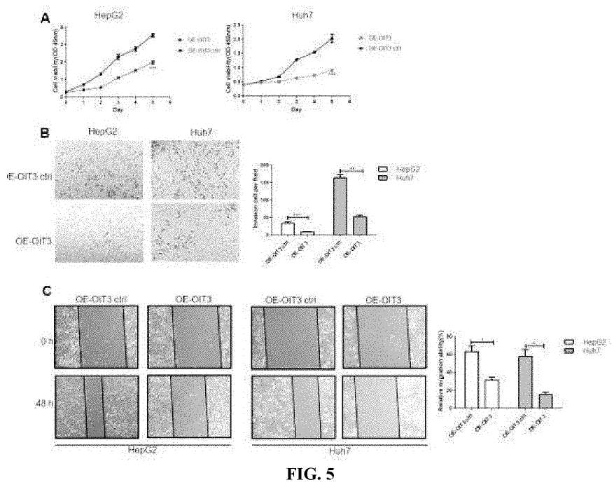

FIG. 5 shows a schematic diagram for verifying the ability of OIT3 to inhibit the proliferation, invasion, and metastasis of hepatoma cells, which indicates that OIT3 plays the role of a tumor suppressor gene in HCC.

Overexpression of OIT3 inhibits the proliferation, migration, and invasion of hepatoma cells.

After overexpression of OIT3,

A: cck8 for the detection of changes in cell proliferation;

B: Transwell for the detection of changes in cell invasiveness;

C: Scratch test for the detection of cell migration (*P<0.05; **P<0.01; ***P<0.001).

Abbreviation: OE-OIT3: Overexpress OIT3; ctrl: Control.

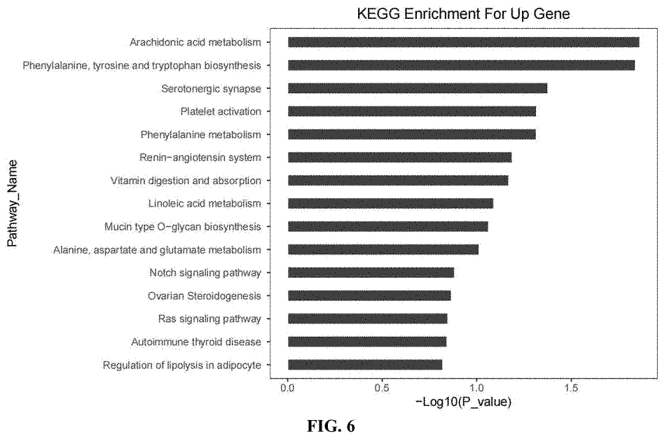

FIG. 6 shows the mechanism of regulation of OIT3 in HCC. It can be seen that many biological mechanisms have changed after the change of OIT3, among which the most obvious is the change in arachidonic acid metabolism, which suggests that OIT3 may be involved in the occurrence and development of HHC by regulating fatty acid metabolism.

This example describes the determination of primer sequences for OIT3 and the expression of OIT3 in HCC, as detected using qRT-PCR.

Primer sequences for OIT3 were obtained from Primer Bank (https://pga/mgh/havard/edu/primerbank/index.html) and were further synthesized into primers.

Forward Primer:
(SEQUENCE ID NO: 1)
TGTTCTGCTTACATCAGCCTG;
Reverse Primer:
(SEQUENCE ID NO: 2)
GTTGTCACATAGAGGAGGACCT

Total RNA Extraction:

After the cells were treated, the culture medium was discarded from the Petri dish, and the Petri dish was washed thrice with PBS. Afterward, 1 mL of TRIZOL was added to the Petri dish and placed on ice for approximately 5-10 min. The cells were then blown down from the Petri dish and transferred to a 1.5 mL EP tube using a pipette. Afterward, 0.2 mL of chloroform (trichloromethane) (⅕ Trizol volume) was added to the EP tube after being placed for 2 min at 25° C.±5° C. with a yellow pipette tip, the EP tube was shaken violently by hand for 15 s, and then placed at 25° C.±5° C. for 5 min before being centrifuged with a high-speed centrifuge at 12000×g at 4° C. for 15 min, and then the delamination was observed.

Next, approximately 0.3 mL of the upper water phase was carefully collected with a yellow pipette tip and then placed in another 1.5 mL inactivated EP tube (while avoiding suctioning the intermediate layer and organic phase).

Approximately 0.3 mL of isopropanol (equal volume) was added to the above-mentioned tubes, fully mixed by shaking the tubes vigorously, and left for 10 min at 25° C.±5° C. (isopropanol was precooled at 4° C. in advance or mixed evenly and then left for 60 min at −20° C., resulting in a better extraction effect) for centrifugation with a high-speed centrifuge at 12000×g at 4° C. for 10 min, and RNA precipitation was observed.

The supernatant was discarded, the residual liquid was sucked from the tube orifice using a filter paper, 1 mL of precooled 75% inactivated ethanol was then added to the tubes, and the tube walls were flicked with the fingers to make RNA precipitate float for washing, followed by centrifugation at 7500×g at 4° C. for 5 min. The resultant supernatant was discarded, and the RNA precipitate was washed again with 75% inactivated ethanol, resulting in the total RNA precipitate after discarding the supernatant. It is worth noting that if the total RNA of HCC tissues is to be extracted, a tissue grinder should be used, and the HCC tissues should be completely ground with grinding beads and TRIZOL.

mRNA Reverse Transcription and mRNA Detection Test

Reagents required: The FastQuant cDNA first strand synthesis kit (KR106) and SuperReal fluorescence quantitative detection kit (FP209) were purchased from Beijing Tiangen Biochemical Co., Ltd., and eight-connected tubes were used for qRT-PCR.

Synthesis of the First Strand of cDNA by a Template of mRNA

Reverse Transcription System 1

| Component | Amount used |
|---|---|
| 5× gDNA Buffer | 2 μL |
| Total RNA | 2 μg |
| RNase-Free ddH2O | Make up to 10 μL |

Reverse Transcription System 2

| Component | Amount used |
|---|---|
| 10× Fast RT Buffer | 2 μL |
| RT Enzyme Mix | 1 μL |
| FQ-RT Primer Mix | 2 μL |
| RNase-Free ddH2O | Make up to 10 μL |

The mix in the reverse transcription reaction was added to the reaction solution of the gDNA removal step, and the mixture was fully mixed. The resulting solution was incubated for 15 min at 42° C., for 3 min at 95° C., and then placed on ice to obtain cDNA.

mRNA Detection Procedure:

Reaction System

| Component | Amount used |
|---|---|
| 2× Talent qPCR PreMix | 10 μL |
| Forward Primer (10 μM) | 0.6 μL |
| Reserve Primer (10 μM) | 0.6 μL |
| cDNA template | 1 |
| 50× ROX Reference DyeΔ | 0.4 μL |
| RNase-Free ddH₂O | Make up to 10 μL |

Real-Time PCR Reaction Procedures

| Phase | Cycle | Temperature | Time | Content |
|---|---|---|---|---|
| pre-denaturation | 1× | 95° C. | 3 min | Initial template |
| PCR reaction | 40× | 95° C. | 5 | denaturation |
| | | | | Template denaturation |
| | | 60° C. | 15 sec | in cycles of PCR |

Annealing/extension
melting curve
analysis (Melting/
Dissociation
Curve Stage)

Summary:

In conclusion, the following conclusions can be made from the above experimental results:

1. OIT3 is expressed in low levels in HCC tissues, and a lower level of expression of OIT3 indicates a worse clinical prognosis in patients;

2. The level of expression of OIT3 in hepatoma cells is lower than that in normal hepatocytes;

3. OIT3 is a cancer suppressor gene, and overexpression of OIT3 can inhibit the proliferation, migration, and invasion of hepatoma cells; and

4. OIT3 can regulate the occurrence and development of HCC by regulating the metabolism of fatty acids (arachidonic acid).

Therefore, the use of the OIT3 gene as a marker for the diagnosis of HCC makes the diagnosis of HCC more accurate and rapid. The OIT3 gene in the present disclosure can provide a new indicator for the diagnosis of HCC, and OIT3 may be a new drug target for the treatment of HCC.

In summary, the tumor marker for HCC in the present disclosure can make the diagnosis of HCC simpler, accurate, and rapid, improve the diagnosis of early HCC, and is suitable for popularization and clinical application.

For those skilled in the art, it is obvious that the present disclosure is not limited to the details of the above exemplary example, and can be implemented in other specific forms without departing from the spirit or basic features of the present disclosure. Thus, from any point of view, the example should be regarded as exemplary and non-limiting. Furthermore, the scope of the present disclosure is defined by appended claims rather than by the above description. Therefore, it is intended to include all variations within the meaning and range of the equivalent elements of the claims in the present disclosure.

In addition, it should be understood that although the Description is described according to the examples, not every embodiment contains an independent technical solution. This description is provided only for the sake of clarity. Those skilled in the art should take the Description as a whole, and the technical solutions in each embodiment can also be properly combined to form other examples that can be understood by those skilled in the art.

1. A novel tumor marker for hepatocellular carcinoma (HCC), wherein the tumor marker for HCC is OIT3, and an expression level of the OIT3 in peripheral blood and HCC tissues of patients with HCC is detected using qRT-PCR;
wherein the OIT3 is significantly lower in HCC tissues and is closely related to the tumor biological behavior of hepatoma cells.

2. The novel tumor marker for HCC according to claim 1, wherein the tumor biological behaviors are proliferation, migration, and invasion.

3. The novel tumor marker for HCC according to claim 2, wherein the tumor marker for HCC, OIT3, is expressed at low levels in HCC tissues, and the expression level thereof is significantly related to the clinical prognosis of patients with HCC.

4. The novel tumor marker for HCC according to claim 1, wherein the tumor marker may be used as a key component of an HCC detection kit by designing primer sequences, and clinical samples of patients with HCC are tested for the tumor marker.

5. The novel tumor marker for HCC according to claim 4, wherein the clinical samples comprise specimens of peripheral blood and HCC tissues, and relevant samples should be collected clinically, wherein the collection procedures of peripheral blood samples are as follows:
collecting 2.5-3.5 mL of fasting venous blood from subjects into a pro-coagulation tube, keeping the same vertically at 25° C.±5° C. for 25-35 min, followed by centrifugation for 8-12 min at 1450-1550 r/min at normal temperature, then transferring the supernatant into a marked EP tube and saving same at −80° C.

6. The novel tumor marker for HCC according to claim 5, wherein the collection procedures of HCC specimens are as follows: cutting fresh HCC tissues and storing same at −80° C. for subsequent extraction of total RNA from serum and HCC tissue samples using TRIzol reagent.

7. The novel tumor marker for HCC, according to claim 6, wherein primer sequences of the OIT3 are as follows:
Forward Primer: TGTTCTGCTTACATCAGCCTG (SEQUENCE ID NO:1);
Reverse Primer: GTTGTCACATAGAGGAGGACCT (SEQUENCE ID NO:2);
and levels of expression of the OIT3 in specimens of HCC tissues and peripheral blood of patients are tested using fluorescence quantitative PCR (qRT-PCR) with specific primers.

8. The novel tumor marker for HCC, according to claim 1, wherein the tumor marker for HCC is applied as a HCC detection and diagnosis reagent and has value in a prediction model for clinical prognosis.

9. An application of detection of the tumor marker for HCC according to claim 1 in the diagnosis of HCC.

10. The application according to claim 9, wherein the application includes early screening of patients with HCC and prediction of clinical prognosis based on the levels of expression of the OIT3, having clinical transformation value.


SEQUENCE LISTING (2 sequences)

SEQ ID NO: 1 - DNA, 21 bp, synthetic construct
  source 1..21: mol_type = other DNA; note = Forward Primer
tgttctgcttacatcagcctg

SEQ ID NO: 2 - DNA, 22 bp, synthetic construct
  source 1..22: mol_type = other DNA; note = Reverse Primer
gttgtcacatagaggaggacct